(19) United States
(12) Patent Application Publication
(10) Pub. No.: US 2008/0132734 A1
(43) Pub. Date: Jun. 5, 2008

(54) Process for making dibutyl ethers from dry 2-butanol

(76) Inventors: Leo Ernest Manzer, Wilmington, DE (US); Michael B. D'Amore, Wilmington, DE (US); Edward S. Miller, Knoxville, TN (US); Jeffrey P. Knapp, Wilmington, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 11/818,355

(22) Filed: Jun. 13, 2007

Related U.S. Application Data
(60) Provisional application No. 60/872,136, filed on Dec. 1, 2006.

(51) Int. Cl. C07C 41/01

(52) U.S. Cl. 568/699

(57) ABSTRACT

The present invention relates to a process for making dibutyl ethers using dry 2-butanol derived from fermentation broth. The dibutyl ethers so produced are useful in transportation fuels.

### CROSS-REFERENCE TO RELATED APPLICATION

This application claims priority under 35 U.S.C. §119 from U.S. Provisional Application Ser. No. 60/872,136 (filed Dec. 1, 2006), the disclosure of which is incorporated by reference herein for all purposes as if fully set forth.

### FIELD OF INVENTION

The present invention relates to a process for making dibutyl ethers using dry 2-butanol obtained from fermentation broth.

### BACKGROUND

Dibutyl ethers are useful as diesel fuel cetane enhancers (R. Kotrba, “Ahead of the Curve”, in Ethanol Producer Magazine, November 2005); an example of a diesel fuel formulation comprising dibutyl ether is disclosed in WO 2001018154. The production of dibutyl ethers from butanol is known (see Karas, L. and Piel, W. J. Ethers, in Kirk-Othmer Encyclopedia of Chemical Technology, Fifth Ed., Vol. 10, Section 5.3, p. 576) and is generally carried out via the dehydration of butanol by sulfuric acid, or by catalytic dehydration over ferric chloride, copper sulfate, silica, or silica-alumina at high temperatures.

Efforts directed at improving air quality and increasing energy production from renewable resources have resulted in renewed interest in alternative fuels, such as ethanol and butanol, that might replace gasoline and diesel fuel. Efforts are currently underway to increase the efficiency of 2-butanol production by fermentative microorganisms utilizing renewable feedstocks, such as corn waste and sugar cane bagasse, as carbon sources. It would be desirable to be able to utilize such 2-butanol streams to produce fuel additives, such as dibutyl ethers.

### SUMMARY

The present invention relates to a process for making at least one dibutyl ether comprising:

(a) obtaining a fermentation broth comprising 2-butanol;

(b) separating dry 2-butanol from said fermentation broth to form separated dry 2-butanol;

(c) contacting the separated dry 2-butanol of step (b), optionally in the presence of a solvent, with at least one acid catalyst at a temperature of about 50 degrees C. to about 450 degrees C. and a pressure from about 0.1 MPa to about 20.7 MPa to produce a reaction product comprising said at least one dibutyl ether; and

(d) recovering said at least one dibutyl ether from said reaction product to obtain at least one recovered dibutyl ether.

The expression “dry 2-butanol” as used in the present specification and claims denotes a material that is predominantly 2-butanol, but may contain small amounts of water (under about 5% by weight relative to the weight of the 2-butanol plus the water), and may contain small amounts of other materials as long as they do not materially affect the catalytic reaction previously described when performed with reagent grade 2-butanol.

The at least one dibutyl ether is useful as a transportation fuel additive.

### BRIEF DESCRIPTION OF THE DRAWING

The Drawing consists of six figures.

FIG. 1 illustrates an overall process useful for carrying out the present invention.

FIG. 2 illustrates a method for producing a 2-butanol stream using distillation wherein fermentation broth comprising 2-butanol and water is used as the feed stream.

FIG. 3 illustrates a method for producing a 2-butanol/water stream using gas stripping wherein fermentation broth comprising 2-butanol and water is used as the feed stream.

FIG. 4 illustrates a method for producing a 2-butanol/water stream using liquid-liquid extraction wherein fermentation broth comprising 2-butanol and water is used as the feed stream.

FIG. 5 illustrates a method for producing a 2-butanol/water stream using adsorption wherein fermentation broth comprising 2-butanol and water is used as the feed stream.

FIG. 6 illustrates a method for producing a 2-butanol/water stream using pervaporation wherein fermentation broth comprising 2-butanol and water is used as the feed stream.

### DETAILED DESCRIPTION

The present invention relates to a process for making at least one dibutyl ether from dry 2-butanol derived from fermentation broth. The at least one dibutyl ether so produced is useful as an additive in transportation fuels, wherein transportation fuels include, but are not limited to, gasoline, diesel fuel and jet fuel.

More specifically, the present invention relates to a process for making at least one dibutyl ether comprising contacting dry 2-butanol with at least one acid catalyst to produce a reaction product comprising at least one dibutyl ether, and recovering said at least one dibutyl ether from said reaction product to obtain at least one recovered dibutyl ether. The “at least one dibutyl ether” is a dibutyl ether, wherein one or both butyl substituents of the ether are selected from the group consisting of 1-butyl, 2-butyl, t-butyl and isobutyl.

The dry 2-butanol reactant for the process of the invention is derived from fermentation broth. One advantage to the microbial (fermentative) production of butanol is the ability to utilize feedstocks derived from renewable sources, such as corn stalks, corn grain, corn cobs, sugar cane, sugar beets or wheat, for the fermentation process. Efforts are currently underway to engineer (through recombinant means) or select for organisms that produce butanol with greater efficiency than is obtained with current microorganisms. Such efforts are expected to be successful, and the process of the present invention will be applicable to any fermentation process that produces 2-butanol at levels currently seen with wild-type microorganisms, or with genetically modified microorganisms from which enhanced production of 2-butanol is obtained.

2-Butanol can be produced by fermentatively producing 2,3-butanediol, followed by converting the 2,3-butanediol chemically to 2-butanol as described in co-filed and commonly owned Patent Application Docket Number CL-3082. According to CL-3082, 2,3-butanediol is converted to 2-butanol by a process comprising contacting a reactant comprising dry or wet 2,3-butanediol, optionally in the presence of at least one inert solvent, with hydrogen in the presence of a catalyst system that can function both as an acid catalyst and as a hydrogenation catalyst at a temperature between about 75 and about 300 degrees Centigrade and a hydrogen pressure between about 345 kPa and about 20.7 MPa, to produce a reaction product comprising 2-butanol; and recovering 2-butanol from the reaction product.

Suitable inert solvents for the conversion of 2,3-butanediol to 2-butanol as described in CL-3082 include liquid hydrocarbons, liquid aromatic compounds, liquid ethers, 2-butanol, and combinations thereof. Preferred solvents include C₅ to C₂₀ straight-chain, branched or cyclic liquid hydrocarbons, C₆ to C₂₀ liquid aromatic compounds, and liquid dialkyl ethers wherein the individual alkyl groups of the dialkyl ether are straight-chain or branched, and wherein the total number of carbons of the dialkyl ether is from 4 to 16.

The 2,3-butanediol (BDO) for the process described in CL-3082 can be obtained by fermentation; microbial fermentation for the production of BDO has been reviewed in detail by Syu, M.-J. (Appl. Microbiol. Biotechnol (2001) 55:10-18). Strains of bacteria useful for producing BDO include Klebsiella pneumoniae and Bacillus polymyxa, as well as recombinant strains of Escherichia coli. Carbon and energy sources, culture media, and growth conditions (such as pH, temperature, aeration and inoculum) are dependent on the microbial strain used, and are described by Ledingham, G. A. and Neish, A. C. (Fermentative production of 2,3-butanediol, in Underkofler, L. A. and Hickey, R. J., Industrial Fermentations, Volume II, Chemical Publishing Co., Inc., New York, 1954, pages 27-93), Garg, S. K. and Jain, A. (Bioresource Technology (1995) 51:103-109), and Syu (supra). These references also describe the use of biomass as the carbon (i. e, sugar) source, as well as the bioreactors and additional fermentation equipment and conditions required for fermentation. One example wherein K. pneumoniae was utilized to produce BDO was provided by Grover, B. S., et al (World J. Microbiol. and Biotech. (1990) 6:328-332). Grover, B. S., et al described the production of BDO using K. pneumoniae NRRL B-199 grown on the reducing sugars in wood hydrolysate. Optimal conditions for a 48 hour fermentation were pH 6.0, a temperature of 30 degrees Centigrade, and 50 grams of reducing sugars per liter of medium.

BDO can be recovered from fermentation broth by a number of techniques well known to those skilled in the art, including distillation, vacuum membrane distillation using a microporous polytetrafluoroethylene membrane and solvent extraction using solvents such as ethyl acetate, diethyl ether, and n-butanol as reviewed by Syu (supra).

The heterogeneous catalyst system useful for the conversion of 2,3-butanediol to 2-butanol as described in CL-3082 is a catalyst system that can function both as an acid catalyst and as a hydrogenation catalyst. The heterogeneous catalyst system can comprise independent catalysts, i.e., at least one solid acid catalyst plus at least one solid hydrogenation catalyst. Alternatively, the heterogeneous catalyst system can comprise a dual function catalyst. A dual function catalyst is defined in CL-3082 as a catalyst wherein at least one solid acid catalyst and at least one solid hydrogenation catalyst are combined into one catalytic material.

Suitable acid catalysts are heterogeneous (or solid) acid catalysts. The at least one solid acid catalyst may be supported on at least one catalyst support (herein referred to as a supported acid catalyst). Solid acid catalysts include, but are not limited to, (1) heterogeneous heteropolyacids (HPAs) and their salts, (2) natural clay minerals, such as those containing alumina or silica (including zeolites), (3) cation exchange resins, (4) metal oxides, (5) mixed metal oxides, (6) metal salts such as metal sulfides, metal sulfates, metal sulfonates, metal nitrates, metal phosphates, metal phosphonates, metal molybdates, metal tungstates, metal borates, and (7) combinations of groups 1 to 6. When present, the metal components of groups 4 to 6 may be selected from elements from Groups I, IIa, IIIa, VIIa, VIIIa, Ib and IIb of the Periodic Table of the Elements, as well as aluminum, chromium, tin, titanium and zirconium.

Preferred solid acid catalysts include cation exchange resins, such as Amberlyst® 15 (Rohm and Haas, Philadelphia, Pa.), Amberlite® 120 (Rohm and Haas), Nafion®, and natural clay materials, including zeolites such as mordenite.

The heterogeneous catalyst system useful for converting 2,3-butanediol to 2-butanol must also comprise at least one solid hydrogenation catalyst. The at least one solid hydrogenation catalyst may be supported on at least one catalyst support (herein referred to as a supported hydrogenation catalyst).

The hydrogenation catalyst may be a metal selected from the group consisting of nickel, copper, chromium, cobalt, rhodium, ruthenium, rhenium, osmium, iridium, platinum, palladium, at least one Raney® metal, platinum black; compounds thereof; and combinations thereof. A promoter such as, without limitation, tin, zinc, copper, gold, silver and combinations thereof may be used to affect the reaction, for example, by increasing activity and catalyst lifetime.

Preferred hydrogenation catalysts include ruthenium, iridium, palladium; compounds thereof; and combinations thereof.

A suitable dual function catalyst can be, but is not limited to, a hydrogenation catalyst comprising a metal selected from the group consisting of nickel, copper, chromium, cobalt, rhodium, ruthenium, rhenium, osmium, iridium, platinum, and palladium; compounds thereof; and combinations thereof; deposited by any means commonly known to those skilled in the art on an acid catalyst selected from the group consisting of (1) heterogeneous heteropolyacids (HPAs) and their salts, (2) natural clay minerals, such as those containing alumina or silica (including zeolites), (3) cation exchange resins, (4) metal oxides, (5) mixed metal oxides, (6) metal salts such as metal sulfides, metal sulfates, metal sulfonates, metal nitrates, metal phosphates, metal phosphonates, metal molybdates, metal tungstates, metal borates, and (7) combinations of groups 1 to 6.

The reaction product comprises 2-butanol, as well as water, and may comprise unreacted BDO and/or methyl ethyl ketone. Dry 2-butanol can be recovered as described below by a refining process that includes at least one distillation step (Doherty, M. F. and M. F. Malone, Conceptual Design of Distillation Systems, McGraw-Hill, New York, 2001) or the use of molecular sieves. As is known to those skilled in the art, molecular sieves are adsorbent materials that have a stronger affinity for one type of atom or molecular in a stream than for other types in the stream. A common use of molecular sieves is the dehydration of ethanol as described, for example in R. L. B. Swain (Molecular sieve dehydrators, how they became the industry standard and how they work, in Jacques, K. A. et al (eds) in The Alcohol Textbook, 3^{rd} Edition, Chapter 19, 1999, Nottingham University Press, U.K.).

2-Butanol for use in the present invention can also be fermentatively produced by recombinant microorganisms as described in copending and commonly owned U.S. Patent Application No. 60/796,816, page 4, line 7 through page 42, line 26, including the sequence listing. In one embodiment, the invention described in 60/796,816 provides a recombinant microbial host cell comprising at least one DNA molecule encoding a polypeptide that catalyzes a substrate to product conversion selected from the group consisting of:

i) pyruvate to alpha-acetolactate

ii) alpha-acetolactate to acetoin

iii) acetoin to 2,3-butanediol

iv) 2,3-butanediol to 2-butanone

v) 2-butanone to 2-butanol

wherein the at least one DNA molecule is heterologous to said microbial host cell and wherein said microbial host cell produces 2-butanol. Methods for generating recombinant microorganisms, including isolating genes, constructing vectors, transforming hosts, and analyzing expression of genes of the biosynthetic pathway are described in detail by Donaldson, et al. in 60/796,816.

Fermentation methodology is well known in the art, and can be carried out in a batch-wise, continuous or semi-continuous manner. As is well known to those skilled in the art, the concentration of 2-butanol in the fermentation broth produced by any process will depend on the microbial strain and the conditions, such as temperature, growth medium, mixing and substrate, under which the microorganism is grown.

Following fermentation, the fermentation broth from the fermentor can be used for the process of the invention. In one preferred embodiment the fermentation broth is subjected to a refining process to produce an aqueous stream comprising an enriched concentration of 2-butanol. By “refining process” is meant a process comprising one unit operation or a series of unit operations that allows for the purification of an impure aqueous stream comprising 2-butanol to yield a stream comprising substantially pure 2-butanol.

Refining processes utilize one or more unit operations, and typically employ at least one distillation step as a means for recovering a fermentation product. It is expected, however, that fermentative processes will produce 2-butanol at very low concentrations relative to the concentration of water in the fermentation broth. This can lead to large capital and energy expenditures to recover the 2-butanol by distillation alone. As such, other techniques can be used in combination with distillation as a means of concentrating the dilute 2-butanol product. In such processes where separation techniques are integrated with the fermentation step, cells can optionally be removed from the stream to be refined by centrifugation or membrane separation techniques, yielding a clarified fermentation broth. These cells are then returned to the fermentor to improve the productivity of the 2-butanol fermentation process. The clarified fermentation broth can then be subjected to techniques such as pervaporation, gas stripping, liquid-liquid extraction, perstraction, adsorption, or combinations thereof. Depending on product mix, these other techniques can provide a stream comprising water and 2-butanol suitable for further purification by distillation to yield a 2-butanol stream.

### Separation of 2-Butanol from Water

1-Butanol and 2-butanol have many common features that allow the separation schemes devised for the separation of 1-butanol and water to be applicable to the 2-butanol and water system. For instance both 1-butanol and 2-butanol are hydrophobic molecules possessing log Kow coefficients of 0.88 and 0.61, respectively. Kow is defined as the partition coefficient of a species at equilibrium in an octanol-water system. Since both 1-butanol and 2-butanol are hydrophobic molecules (Kow=7.6 and 4.1, respectively), one would expect both molecules to favorably partition into a separate non-aqueous phase such as decanol or adsorb onto various hydrophobic solid phases such as silicone or silicalite. In this regard liquid-liquid extraction and adsorption are viable separation options for 2-butanol from water.

In addition, both 1-butanol and 2-butanol are relatively volatile molecules at dilute concentration and have favorable K values, or vapor-liquid partition coefficients, relative to ethanol, when in solution with water. Another useful thermodynamic term is α, or relative volatility, which is the ratio of partition coefficients, K values, for a given binary system. For a given concentration and temperature less than 100° C., the values for K and α are greater for 2-butanol vs. 1-butanol in their respective butanol-water systems, i.e. 5.3 vs 4.6, and 43 vs 37, respectively. This indicates that in evaporative separation schemes such as gas stripping, pervaporation, and distillation, 2-butanol should separate more efficiently from water than 1-butanol from water at a given temperature. At 100° C. the K and α values are very similar between 2-butanol and 1-butanol, 31 vs 30, and 31 vs. 30, respectively, indicating that separation processes based on evaporative means and designed for operation in this temperature range should perform with equal efficiency.

The separation of 1-butanol from water, and the separation of 1-butanol from a mixture of acetone, ethanol, 1-butanol and water by distillation have been described as part of the ABE fermentation process literature. In particular, in a 1-butanol and water system, 1-butanol forms a low boiling heterogeneous azeotrope in equilibrium with 2 liquid phases comprised of 1-butanol and water. This azeotrope is formed at a vapor phase composition of approximately 58% by weight 1-butanol (relative to the weight of water plus 1-butanol) when the system is at atmospheric pressure (as described by Doherty, M. F. and Malone, M. F. in Conceptual Design of Distillation Systems (2001), Chapter 8, pages 365-366, McGraw-Hill, New York). The liquid phases are roughly 6% by weight 1-butanol (relative to the weight of water plus 1-butanol) and 80% by weight 1-butanol (relative to the weight of water plus 1-butanol), respectively.

Unlike 1-butanol, 2-butanol forms a minimum boiling homogeneous azeotrope with water. In this regard 2-butanol behaves more like ethanol than 1-butanol. In the 2-butanol-water system the vapor phase is in equilibrium with a single liquid phase of the same composition. The azeotrope is formed at a vapor phase composition of 73% by weight 2-butanol (relative to the weight of water plus 2-butanol) (as described by Doherty, M. F. and Malone, M. F. in Conceptual Design of Distillation Systems (2001), Chapter 8, pages 365-366, McGraw-Hill, New York). Although the high relative volatility of 2-butanol over water makes distillation an attractive separations option, the homogeneous azeotrope provides a boundary to further increasing the purity of the butanol product stream by simple distillation. In systems where homogeneous azeotropes are present, a separate component can be added to modify the separation characteristics of the material to be separated from the bulk medium. The added component is typically called an entrainer and the process of distillation using the entrainer referred to as extractive distillation. Such systems have been described for separating 2-butanol from water and are mentioned by reference below.

### Distillation

Before molecular sieves came into use, ethanol was commonly purified using azeotropic distillation with a specially chosen entrainer. Some of the entrainers used or proposed for the ethanol separation included benzene, cyclohexane, iso-octane, pentane, carbon tetrachloride, trichloroethylene, diethyl ether, 1-butanol, and ethyl acetate as generally described in Doherty, M. F. and M. F. Malone, Conceptual Design of Distillation Systems, McGraw-Hill, New York, 2001. The commercial process for making 2-butanol from n-butylenes uses azeotropic distillation to remove impurities, including water. The separation scheme underpinning the commercial 2-butanol process has been described by Takaoka, S., Acetone, Methyl Ethyl Ketone, and Methyl Isobutyl Ketone, Report No. 77, Process Economics Program, Stanford Research Institute, Menlo Park, Calif., May 1972; Kovach III, J. W. and W. D. Seider, “Heterogeneous Azeotropic Distillation: Experimental and Simulation Results,” AlChE J., 33(8), 1300-1314, 1987; Kovach III, J. W. and W. D. Seider, “Vapor-Liquid and Liquid-Liquid Equilibria for the System sec-Butyl Alcohol-Di-sec-Butyl Ether-Water,” J. Chem. Eng. Data, 33, 16-20, 1988; and Baumann, G. P., “Secondary Butanol Purification Process”, U.S. Pat. No. 3,203,872, Aug. 31, 1965. In the latter example, the entrainer used is a reaction byproduct (di-sec-butyl ether) already in the feed to the column.

A pure 2-butanol stream derived from aqueous fermentation broth containing 2-butanol can be obtained by a similar extractive distillation process as described in the references above. In design of the distillation scheme careful selection is needed for the entrainer to be used in the process. A successful entrainer must form one or more binary and/or ternary azeotropes with water and possibly 2-butanol that has a boiling point lower than the 2-butanol-water azeotrope. This way the entrainer-containing azeotrope(s) will distill overhead. The boiling point of the entrainer is not required to be below that of the 2-butanol-water azeotrope, only its azeotropes must be. The azeotropes formed by the entrainer should also be heterogeneous so that decantation can be used to cross the azeotropes and distillation boundaries. It is preferable that the entrainer has very low solubility with water. Additionally, the composition of the feed to the azeotropic distillation column can affect the feasibility and/or design of the process. Many of the compounds known to work for ethanol dehydration are also likely to work as entrainers for 2-butanol. A specific embodiment of the current invention uses toluene as the entrainer in an extractive distillation process. The example is not meant to be limiting of the current invention but rather descriptive.

A three-column extractive distillation process can be employed for recovering 2-butanol from water. In such a process the first distillation column is used to enrich the 2-butanol in the overhead stream to near its azeotropic composition, thus reducing the water content and mass of the stream to be sent on to the extractive distillation column system. This stream is then cooled and fed to a second azeotropic distillation column in which a toluene-rich entrainer stream is also fed. Toluene meets the necessary criteria outlined above to be used as an entrainer in an extractive distillation system. It forms minimum boiling azeotropes with water and 2-butanol, respectively, and also forms a ternary minimum boiling azeotrope with 2-butanol and water together. The boiling point of the ternary azeotrope is below that of the other azeotropes. Addition of the toluene entrainer stream effectively moves the overall composition of the feed to the azeotropic distillation column across the distillation boundary set by the 2-butanol/water and toluene/2-butanol/water ternary azeotropes. In the azeotropic distillation column 2-butanol can be obtained as a bottoms product stream. The butanol product stream coming from the azeotropic column can then be used directly as the reactant for the process of the present invention. The overhead stream from the azeotropic distillation column is a vaporous ternary 2-butanol/toluene/water azeotrope. The composition of the ternary azeotrope lies within the immiscibility region of the aqueous toluene/water/2-butanol phase equilibria. Thus, the ternary azeotrope formed is a minimum boiling heterogeneous azeotrope. Advantage can be taken of this system by simple subcooling of the overhead stream into the 2 phase region. Subcooling into this region allows the mixture to split into two phases which can be decanted. The top phase, an organic toluene-rich phase, is the source of the toluene feed to the azeotropic distillation column. The bottom phase, a toluene-lean aqueous phase, is sent to a third distillation column to recover the residual toluene and to further remove water from the system.

### Pervaporation

Generally, there are two steps involved in the removal of volatile components by pervaporation. One is the sorption of the volatile component into the membrane, and the other is the diffusion of the volatile component through the membrane due to a concentration gradient. The concentration gradient is created either by a vacuum applied to the opposite side of the membrane or through the use of a sweep gas, such as air or carbon dioxide, also applied along the backside of the membrane. Pervaporation for the separation of 1-butanol from a fermentation broth has been described by Meagher, M. M., et al in U.S. Pat. No. 5,755,967 (Column 5, line 20 through Column 20, line 59) and by Liu, F., et al (Separation and Purification Technology (2005) 42:273-282). According to U.S. Pat. No. 5,755,967, acetone and/or 1-butanol were selectively removed from an ABE fermentation broth using a pervaporation membrane comprising silicalite particles embedded in a polymer matrix. Examples of polymers include polydimethylsiloxane and cellulose acetate, and vacuum was used as the means to create the concentration gradient. The method of U.S. Pat. No. 5,755,967 can similarly be used to recover a stream comprising 2-butanol and water from fermentation broth, and this stream can be further treated by distillation to produce a 2-butanol stream that can be used as the reactant of the present invention.

### Gas Stripping

In general, gas stripping refers to the removal of volatile compounds, such as butanol, from fermentation broth by passing a flow of stripping gas, such as carbon dioxide, helium, hydrogen, nitrogen, or mixtures thereof, through the fermentor culture or through an external stripping column to form an enriched stripping gas. Gas stripping to remove 1-butanol during the ABE fermentation process has been exemplified by Ezeji, T., et al (U.S. Patent Application No. 2005/0089979, paragraphs 16 through 84). According to U.S. 2005/0089979, a stripping gas (carbon dioxide and hydrogen) was fed into a fermentor via a sparger. The flow rate of the stripping gas through the fermentor was controlled to give the desired level of solvent removal. The flow rate of the stripping gas is dependent on such factors as configuration of the system, cell concentration and solvent concentration in the fermentor. This process can also be used to produce an enriched stripping gas comprising 2-butanol and water, and this stream can be further treated by distillation to produce a 2-butanol stream that can be used as the reactant of the present invention.

### Adsorption

Using adsorption, organic compounds of interest are removed from dilute aqueous solutions by selective sorption of the organic compound by a sorbant, such as a resin. Feldman, J. in U.S. Pat. No. 4,450,294 (Column 3, line 45 through Column 9, line 40 (Example 6)) describes the recovery of an oxygenated organic compound from a dilute aqueous solution with a cross-linked polyvinylpyridine resin or nuclear substituted derivative thereof. Suitable oxygenated organic compounds included ethanol, acetone, acetic acid, butyric acid, n-propanol and n-butanol. The adsorbed compound was desorbed using a hot inert gas such as carbon dioxide. This process can also be used to recover an aqueous stream comprising desorbed 2-butanol, and this stream can be further treated by distillation to produce a 2-butanol stream that can be used as the reactant of the present invention.

### Liquid-Liquid Extraction

Liquid-liquid extraction is a mass transfer operation in which a liquid solution (the feed) is contacted with an immiscible or nearly immiscible liquid (solvent) that exhibits preferential affinity or selectivity towards one or more of the components in the feed, allowing selective separation of said one or more components from the feed. The solvent comprising the one or more feed components can then be separated, if necessary, from the components by standard techniques, such as distillation or evaporation. One example of the use of liquid-liquid extraction for the separation of butyric acid and butanol from microbial fermentation broth has been described by Cenedella, R. J. in U.S. Pat. No. 4,628,116 (Column 2, line 28 through Column 8, line 57). According to U.S. Pat. No. 4,628,116, fermentation broth containing butyric acid and/or butanol was acidified to a pH from about 4 to about 3.5, and the acidified fermentation broth was then introduced into the bottom of a series of extraction columns containing vinyl bromide as the solvent. The aqueous fermentation broth, being less dense than the vinyl bromide, floated to the top of the column and was drawn off. Any butyric acid and/or butanol present in the fermentation broth was extracted into the vinyl bromide in the column. The column was then drawn down, the vinyl bromide was evaporated, resulting in purified butyric acid and/or butanol.

Other solvent systems for liquid-liquid extraction, such as decanol, have been described by Roffler, S. R., et al. (Bioprocess Eng. (1987) 1:1-12) and Taya, M., et al (J. Ferment. Technol. (1985) 63:181). In these systems, two phases were formed after the extraction: an upper less dense phase comprising decanol, 1-butanol and water, and a more dense phase comprising mainly decanol and water. Aqueous 1-butanol was recovered from the less dense phase by distillation.

These extractive processes can also be used to obtain a stream comprising 2-butanol that can be further treated by distillation to produce a 2-butanol stream that can be used as the reactant of the present invention.

The dry 2-butanol stream as obtained by any of the above methods can be the reactant for the process of the present invention. The reaction to form at least one dibutyl ether is performed at a temperature of from about 50 degrees Centigrade to about 450 degrees Centigrade. In a more specific embodiment, the temperature is from about 100 degrees Centigrade to about 250 degrees Centigrade.

The reaction can be carried out under an inert atmosphere at a pressure of from about atmospheric pressure (about 0.1 MPa) to about 20.7 MPa. In a more specific embodiment, the pressure is from about 0.1 MPa to about 3.45 MPa. Suitable inert gases include nitrogen, argon and helium.

The reaction can be carried out in liquid or vapor phase and can be run in either batch or continuous mode as described, for example, in H. Scott Fogler, (Elements of Chemical Reaction Engineering, 2^{nd} Edition, (1992) Prentice-Hall Inc, CA).

The at least one acid catalyst can be a homogeneous or heterogeneous catalyst. Homogeneous catalysis is catalysis in which all reactants and the catalyst are molecularly dispersed in one phase. Homogeneous acid catalysts include, but are not limited to inorganic acids, organic sulfonic acids, heteropolyacids, fluoroalkyl sulfonic acids, metal sulfonates, metal trifluoroacetates, compounds thereof and combinations thereof. Examples of homogeneous acid catalysts include sulfuric acid, fluorosulfonic acid, phosphoric acid, p-toluenesulfonic acid, benzenesulfonic acid, hydrogen fluoride, phosphotungstic acid, phosphomolybdic acid, and trifluoromethanesulfonic acid.

Heterogeneous catalysis refers to catalysis in which the catalyst constitutes a separate phase from the reactants and products.

Heterogeneous acid catalysts include, but are not limited to 1) heterogeneous heteropolyacids (HPAs), 2) natural clay minerals, such as those containing alumina or silica, 3) cation exchange resins, 4) metal oxides, 5) mixed metal oxides, 6) metal salts such as metal sulfides, metal sulfates, metal sulfonates, metal nitrates, metal phosphates, metal phosphonates, metal molybdates, metal tungstates, metal borates, and 7) zeolites, 8) combinations of groups 1-7. See, for example, Solid Acid and Base Catalysts, pages 231-273 (Tanabe, K., in Catalysis: Science and Technology, Anderson, J. and Boudart, M (eds.) 1981 Springer-Verlag, New York) for a description of solid catalysts.

The heterogeneous acid catalyst may also be supported on a catalyst support. A support is a material on which the acid catalyst is dispersed. Catalyst supports are well known in the art and are described, for example, in Satterfield, C. N. (Heterogeneous Catalysis in Industrial Practice, 2^{nd} Edition, Chapter 4 (1991) McGraw-Hill, New York).

One skilled in the art will know that conditions, such as temperature, catalytic metal, support, reactor configuration and time can affect the reaction kinetics, product yield and product selectivity. Depending on the reaction conditions, such as the particular catalyst used, products other than dibutyl ethers may be produced when 2-butanol is contacted with an acid catalyst. Additional products comprise butenes and isooctenes. Standard experimentation, performed as described in the Examples herein, can be used to optimize the yield of dibutyl ether from the reaction.

Following the reaction, if necessary, the catalyst can be separated from the reaction product by any suitable technique known to those skilled in the art, such as decantation, filtration, extraction or membrane separation (see Perry, R. H. and Green, D. W. (eds), Perry's Chemical Engineer's Handbook, 7^{th} Edition, Section 13, 1997, McGraw-Hill, New York, Sections 18 and 22).

The at least one dibutyl ether can be recovered from the reaction product by distillation as described in Seader, J. D., et al (Distillation, in Perry, R. H. and Green, D. W. (eds), Perry's Chemical Engineer's Handbook, 7^{th} Edition, Section 13, 1997, McGraw-Hill, New York). Alternatively, the at least one dibutyl ether can be recovered by phase separation, or extraction with a suitable solvent, such as trimethylpentane or octane, as is well known in the art. Unreacted 2-butanol can be recovered following separation of the at least one dibutyl ether and used in subsequent reactions. The at least one recovered dibutyl ether can be added to a transportation fuel as a fuel additive.

The present process and certain embodiments for accomplishing it are shown in greater detail in the Drawing figures.

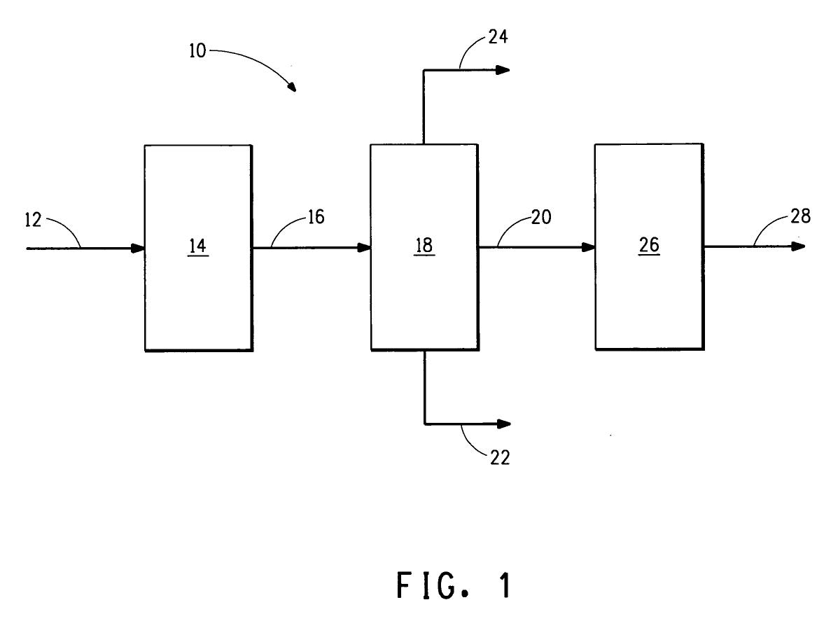

Referring now to FIG. 1, there is shown a block diagram illustrating in a very general way apparatus 10 for deriving dibutyl ether from 2-butanol produced by fermentation. An aqueous stream 12 of biomass-derived carbohydrates is introduced into a fermentor 14. The fermentor 14 contains at least one microorganism (not shown) capable of fermenting the carbohydrates to produce a fermentation broth that comprises 2-butanol and water. A stream 16 of the fermentation broth is introduced into refining apparatus 18 in order to make a stream of dry 2-butanol. The dry 2-butanol is removed from the refining apparatus 18 as stream 20. Water is removed from the refining apparatus 18 as stream 22. Other organic components present in the fermentation broth may be removed as stream 24. The dry 2-butanol stream 20 is introduced into reaction vessel 26 containing an acid catalyst (not shown) capable of converting the 2-butanol into a reaction product comprising at least one dibutyl ether. The reaction product is removed as stream 28.

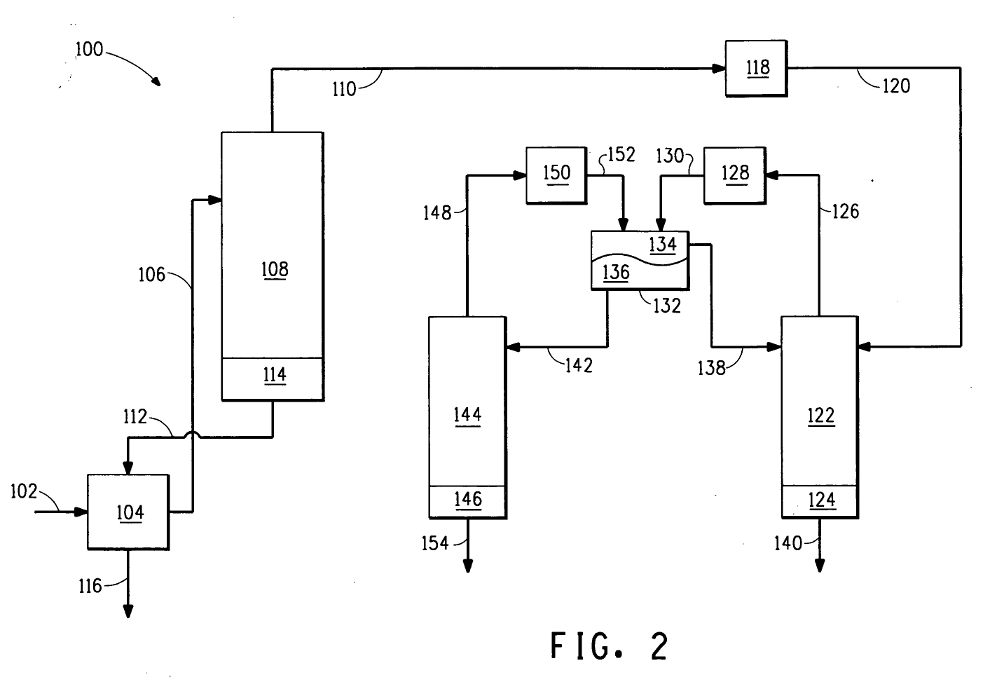

Referring now to FIG. 2, there is shown a block diagram for refining apparatus 100, suitable for producing a 2-butanol stream, when the fermentation broth comprises 2-butanol and water. A stream 102 of fermentation broth is introduced into a feed preheater 104 to raise the broth to a temperature of approximately 95° C. to produce a heated feed stream 106 which is introduced into a beer column 108. The design of the beer column 108 needs to have a sufficient number of theoretical stages to cause separation of 2-butanol from water such that an 2-butanol/water azeotrope can be removed as a vaporous 2-butanol/water azeotrope overhead stream 110 and hot water as a bottoms stream 112. Bottoms stream 112 is used to supply heat to feed preheater 104 and leaves feed preheater 104 as a lower temperature bottoms stream 116. Reboiler 114 is used to supply heat to beer column 108. Vaporous 2-butanol/water azeotrope overhead stream 110 is roughly 73% by weight relative to the total weight of the 2-butanol plus water in the stream. Vaporous 2-butanol/water azeotrope stream 110 can be fed to condenser 118, which lowers the stream temperature causing the vaporous 2-butanol/water azeotrope overhead stream 110 to condense into liquid 2-butanol/water azeotrope stream 120 of the same composition. Liquid 2-butanol/water azeotrope stream 120 is then fed into azeotropic column 122 that is equipped with reboiler 124 to provide necessary heat for the column. Azeotropic column 122 contains a sufficient number of theoretical stages necessary to effect the separation of 2-butanol as a bottoms product from a ternary azeotropic mixture of 2-butanol, toluene, and water. In azeotropic column 122, toluene is added as toluene-rich organic stream 138 which alters the composition of liquid 2-butanol/water azeotrope stream 120 fed to azeotropic column 122 and allows for butanol recovery as butanol stream 140 leaving the bottom of azeotropic column 122. Butanol stream 140 can then be used as the feed stream to a reaction vessel (not shown) in which the 2-butanol is catalytically converted to a reaction product that comprises at least one dibutyl ether. Leaving the top of azeotropic column 122 is vaporous toluene ternary azeotropic stream 126, which is then fed to condenser 128 which lowers the temperature of vaporous toluene ternary azeotropic stream 126 causing it to condense into biphasic toluene ternary azeotropic stream 130, which is then fed into decanter 132. Decanter 132 will contain a toluene-lean aqueous phase 136 that is approximately 95% by weight water, approximately 5% by weight 2-butanol, and less than approximately 1% by weight toluene. Decanter 132 will also contain an upper toluene-rich organic phase 134 that is approximately 47% by weight 2-butanol, approximately 28% by weight water, and approximately 25% by weight toluene. Toluene-rich organic stream 138 of upper toluene-rich organic phase 134 is introduced near the top of azeotropic column 122 to provide reflux for this column. Leaving the bottom of decanter 132 is toluene-lean aqueous stream 142 which is fed to entrainer recovery column 144 equipped with reboiler 146 to provide necessary heat for the column. Entrainer recovery column 144 contains a sufficient number of theoretical stages necessary to effect the separation of water from a ternary azeotropic mixture 2-butanol, toluene, and water. Leaving the top of entrainer recovery column 144 is vaporous toluene ternary azeotropic stream 148 which is fed to condenser 150 which lowers the temperature of vaporous toluene ternary azeotropic stream 148 causing it to condense into biphasic toluene ternary azeotropic stream 152 which is then fed into decanter 132. Leaving the bottom of entrainer recovery column 144 is water stream 154.

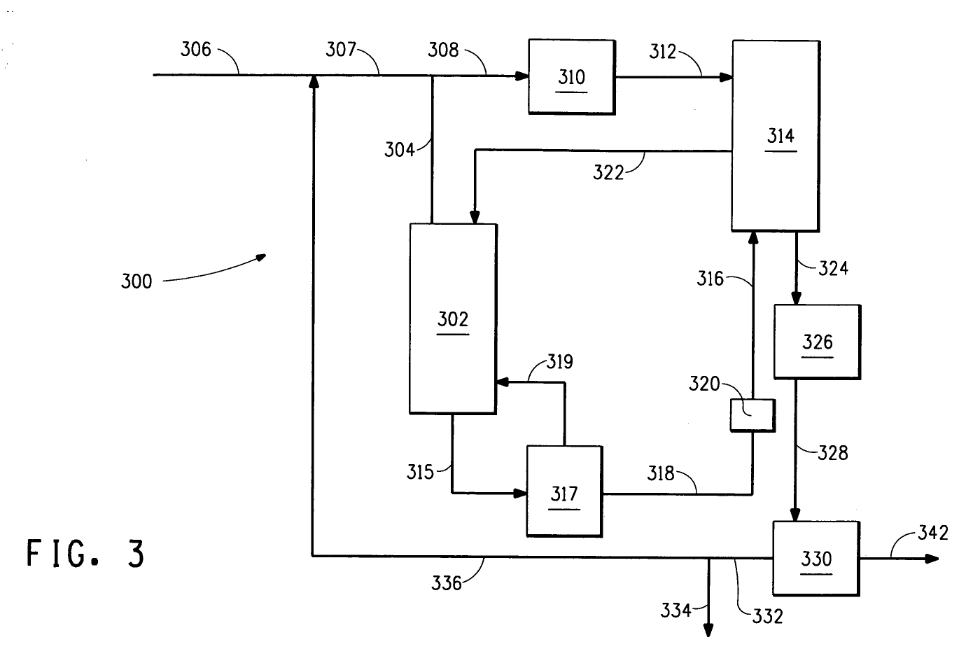

Referring now to FIG. 3, there is shown a block diagram for refining apparatus 300, suitable for producing a dryable aqueous 2-butanol stream when the fermentation broth comprises 2-butanol and water. Fermentor 302 contains a fermentation broth comprising liquid 2-butanol and water and a gas phase comprising CO₂ and to a lesser extent some vaporous 2-butanol and water. A CO₂ stream 304 is then mixed with combined CO₂ stream 307 to give second combined CO₂ stream 308. Second combined CO₂ stream 308 is then fed to heater 310 and heated to 60° C. to give heated CO₂ stream 312. Heated CO₂ stream is then fed to gas stripping column 314 where it is brought into contact with heated clarified fermentation broth stream 316. Heated clarified fermentation broth stream 316 is obtained by heating clarified broth stream 318 to 50° C. in heater 320. Clarified fermentation broth stream 318 is obtained following separation of cells in cell separator 317. Also leaving cell separator 317 is concentrated cell stream 319 that is recycled directly to fermentor 302. The feed stream 315 to cell separator 317 comprises the liquid phase of fermentor 302. Gas stripping column 314 contains a sufficient number of theoretical stages necessary to effect the transfer of 2-butanol from the liquid phase to the gas phase. The number of theoretical stages is dependent on the contents of both streams 312 and 316, as well as their flow rates and temperatures. Leaving gas stripping column 314 is a 2-butanol depleted clarified fermentation broth stream 322 that is recirculated to fermentor 302. A 2-butanol enriched gas stream 324 leaving gas stripping column 314 is then fed to compressor 326, where it is compressed. Following compression, a compressed gas stream 328 comprising 2-butanol is then fed to condenser 330 where the 2-butanol in the gas stream is condensed into a liquid phase that is separate from non-condensable components in the stream 328. Leaving the condenser 330 is 2-butanol depleted gas stream 332. A first portion of gas stream 332 is bled from the system as bleed gas stream 334, and the remaining second portion of 2-butanol depleted gas stream 332, stream 336, is then mixed with makeup CO₂ gas stream 306 to form combined CO₂ gas stream 307. The condensed 2-butanol phase in condenser 330 leaves as aqueous 2-butanol stream 342 and can be used as the feed to a distillation apparatus for further dehydration of the aqueous 2-butanol stream to produce a dry 2-butanol stream, which can then be used as the feed to a reaction vessel in which dry 2-butanol is catalytically converted to a reaction product that comprises at least one dibutyl ether.

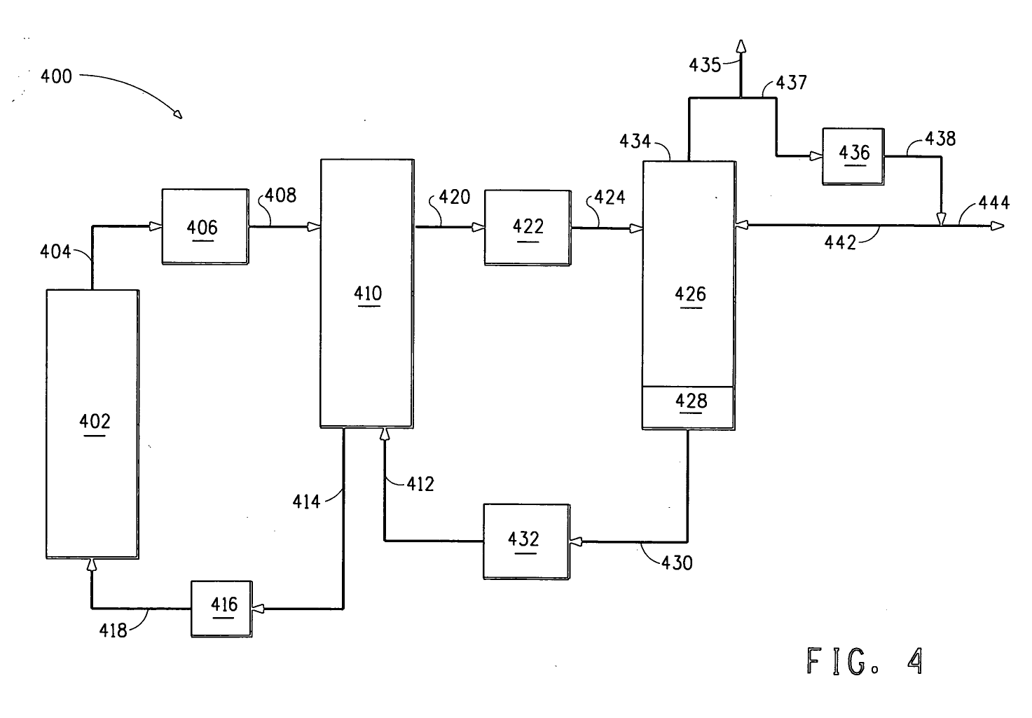

Referring now to FIG. 4, there is shown a block diagram for refining apparatus 400, suitable for producing a dryable aqueous 2-butanol stream, when the fermentation broth comprises 2-butanol and water. Fermentor 402 contains a fermentation broth comprising 2-butanol and water and a gas phase comprising CO₂ and to a lesser extent some vaporous 2-butanol and water. A stream 404 of fermentation broth is introduced into a feed preheater 406 to raise the broth temperature to produce a heated fermentation broth stream 408 which is introduced into solvent extractor 410. In solvent extractor 410, heated fermentation broth stream 408 is brought into contact with cooled solvent stream 412, the solvent used in this case being decanol. Leaving solvent extractor 410 is raffinate stream 414 that is depleted in 2-butanol. Raffinate stream 414 is introduced into raffinate cooler 416 where it is lowered in temperature and returned to fermentor 402 as cooled raffinate stream 418. Also leaving solvent extractor 410 is extract stream 420 that comprises solvent, 2-butanoland water. Extract stream 420 is introduced into solvent heater 422 where it is heated. Heated extract stream 424 is then introduced into solvent recovery distillation column 426, where the solvent is caused to separate from the 2-butanol and water. Solvent column 426 is equipped with reboiler 428 necessary to supply heat to solvent column 426. Leaving the bottom of solvent column 426 is solvent stream 430. Solvent stream 430 is then introduced into solvent cooler 432 where it is cooled to 50° C. Cooled solvent stream 412 leaves solvent cooler 432 and is returned to extractor 410. Leaving the top of solvent column 426 is solvent overhead stream 434 that comprises an azeotropic mixture of 2-butanol and water with trace amounts of solvent. This represents the first substantially concentrated and partially purified 2-butanol/water stream where a portion of the stream (azeotropic vapor stream 435) could be fed to a distillation apparatus (not shown) for further dehydration to produce a dry 2-butanol stream for use as the feed to a reaction vessel in which dry 2-butanol is catalytically converted to a reaction product comprising at least one butene. The remaining portion of solvent overhead stream 434 (stream 437) is then fed into condenser 436 where the vaporous solvent overhead stream is caused to condense into a liquid stream 438 of similar composition. Stream 438 is then optionally split into 2 streams depending on if azeotropic vapor stream 435 is used as the feed for further dehydration by distillation to produce a dry 2-butanol stream. Reflux stream 442 is sent back to solvent column 426 to provide rectification. If azeotropic vapor stream 435 is not used as a feed stream for the process of the invention, optional intermediate product stream 444 can be introduced as the feed to a distillation apparatus (not shown) that is capable of further dehydrating the aqueous 2-butanol stream to produce a dry 2-butanol stream for use as the feed to a reaction vessel in which dry 2-butanol is catalytically converted to a reaction product comprising at least one dibutyl ether.

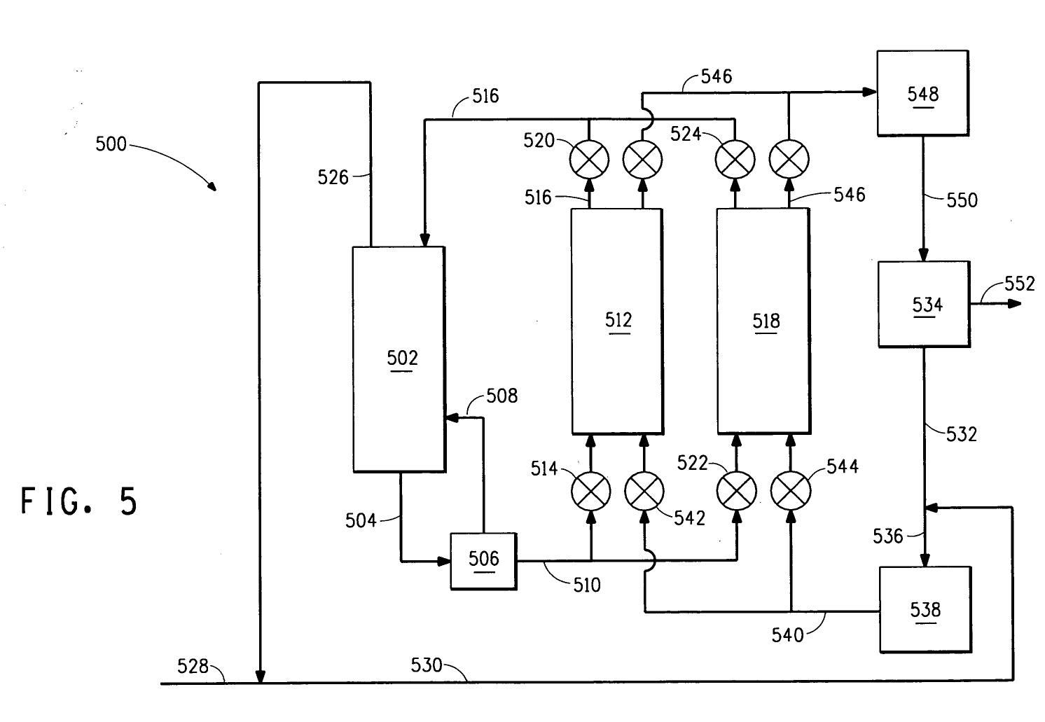

Referring now to FIG. 5, there is shown a block diagram for refining apparatus 500, suitable for concentrating 2-butanol, when the fermentation broth comprises 2-butanol and water. Fermentor 502 contains a fermentation broth comprising 2-butanol and water and a gas phase comprising CO₂ and to a lesser extent some vaporous 2-butanol and water. A 2-butanol-containing fermentation broth stream 504 leaving fermentor 502 is introduced into cell separator 506. Cell separator 506 can be comprised of centrifuges or membrane units to accomplish the separation of cells from the fermentation broth. Leaving cell separator 506 is cell-containing stream 508 which is recycled back to fermentor 502. Also leaving cell separator 506 is clarified fermentation broth stream 510. Clarified fermentation broth stream 510 is then introduced into one or a series of adsorption columns 512 where the 2-butanol is preferentially removed from the liquid stream and adsorbed on the solid phase adsorbent (not shown). Diagrammatically, this is shown in FIG. 5 as a two adsorption column system, although more or fewer columns could be used. The flow of clarified fermentation broth stream 510 is directed to the appropriate adsorption column 512 through the use of switching valve 514. Leaving the top of adsorption column 512 is 2-butanol depleted stream 516 which passes through switching valve 520 and is returned to fermentor 502. When adsorption column 512 reaches capacity, as evidenced by an increase in the 2-butanol concentration of the 2-butanol depleted stream 516, flow of clarified fermentation broth stream 510 is then directed through switching valve 522 by closing switching valve 514. This causes the flow of clarified fermentation broth stream 510 to enter second adsorption column 518 where the 2-butanol is adsorbed onto the adsorbent (not shown). Leaving the top of second adsorption column 518 is a 2-butanol depleted stream that is essentially the same as 2-butanol depleted stream 516. Switching valves 520 and 524 perform the function to divert flow of depleted 2-butanol stream 516 from returning to one of the other columns that is currently being desorbed. When either adsorption column 512 or second adsorption column 518 reaches capacity, the 2-butanol and water adsorbed into the pores of the adsorbent must be removed. This is accomplished using a heated gas stream to effect desorption of adsorbed 2-butanol and water. The CO₂ stream 526 leaving fermentor 502 is first mixed with makeup gas stream 528 to produce combined gas stream 530. Combined gas stream 530 is then mixed with the cooled gas stream 532 leaving decanter 534 to form second combined gas stream 536. Second combined gas stream 536 is then fed to heater 538. Leaving heater 538 is heated gas stream 540 which is diverted into one of the two adsorption columns through the control of switching valves 542 and 544. When passed through either adsorption column 512 or second adsorption column 518, heated gas stream 540 removes the 2-butanol and water from the solid adsorbent. Leaving either adsorption column is 2-butanol/water rich gas stream 546. 2-Butanol/water rich gas stream 546 then enters gas chiller 548 which causes the vaporous 2-butanol and water in 2-butanol/water rich gas stream 546 to condense into a liquid phase that is separate from the other noncondensable species in the stream. Leaving gas chiller 548 is a biphasic gas stream 550 which is fed into decanter 534. In decanter 534 the condensed 2-butanol/water phase is separated from the gas stream. Leaving decanter 534 is an aqueous 2-butanol stream 552 which is then fed to a distillation apparatus (not shown) that is capable of further dehydrating the aqueous 2-butanol stream to produce a dry 2-butanol stream for use as the feed to a reaction vessel in which dry 2-butanol is catalytically converted to a reaction product comprising at least one dibutyl ether. Also leaving decanter 534 is cooled gas stream 532.

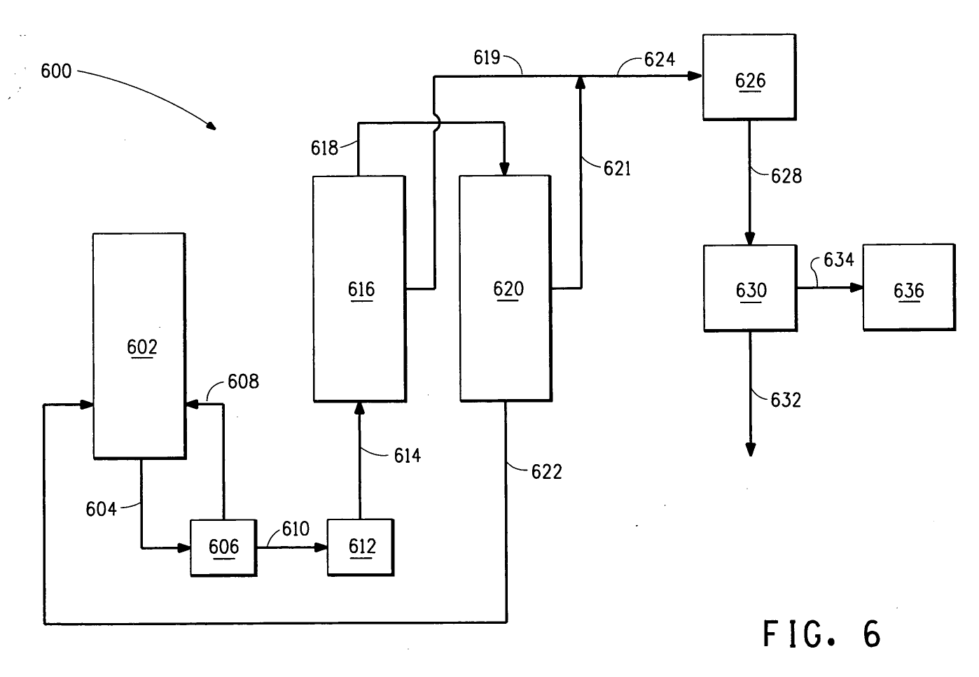

Referring now to FIG. 6, there is shown a block diagram for refining apparatus 600, suitable for producing a dryable aqueous 2-butanol stream, when the fermentation broth comprises 2-butanol and water. Fermentor 602 contains a fermentation broth comprising 2-butanol and water and a gas phase comprising CO₂ and to a lesser extent some vaporous 2-butanol and water. A 2-butanol-containing fermentation broth stream 604 leaving fermentor 602 is introduced into cell separator 606. 2-Butanol-containing stream 604 may contain some non-condensable gas species, such as carbon dioxide. Cell separator 606 can be comprised of centrifuges or membrane units to accomplish the separation of cells from the fermentation broth. Leaving cell separator 606 is concentrated cell stream 608 that is recycled back to fermentor 602. Also leaving cell separator 606 is clarified fermentation broth stream 610. Clarified fermentation broth stream 610 can then be introduced into optional heater 612 where it is optionally raised to a temperature of 40 to 80° C. Leaving optional heater 612 is optionally heated clarified broth stream 614. Optionally heated clarified broth stream 614 is then introduced to the liquid side of first pervaporation module 616. First pervaporation module 616 contains a liquid side that is separated from a low pressure or gas phase side by a membrane (not shown). The membrane serves to keep the phases separated and also exhibits a certain affinity for 2-butanol. In the process of pervaporation any number of pervaporation modules can be used to effect the separation. The number is determined by the concentration of species to be removed and the size of the streams to be processed. Diagrammatically, two pervaporation units are shown in FIG. 6, although any number of units can be used. In first pervaporation module 616, 2-butanol is selectively removed from the liquid phase through a concentration gradient caused when a vacuum is applied to the low pressure side of the membrane. Optionally a sweep gas can be applied to the non-liquid side of the membrane to accomplish a similar purpose. The first depleted 2-butanol stream 618 exiting first pervaporation module 616 then enters second pervaporation module 620. Second 2-butanol depleted stream 622 exiting second pervaporation module 620 is then recycled back to fermentor 602. The low pressure streams 619, 621 exiting first and second pervaporation modules 616 and 620, respectively, are combined to form low pressure 2-butanol/water stream 624. Low pressure 2-butanol/water stream 624 is then fed into cooler 626 where the 2-butanol and water in low pressure 2-butanol/water stream 624 is caused to condense. Leaving cooler 626 is condensed low pressure 2-butanol/water stream 628. Condensed low pressure 2-butanol/water stream 628 is then fed to receiver vessel 630 where the condensed 2-butanol/water stream collects and is withdrawn as stream 632. Vacuum pump 636 is connected to the receiving vessel 630 by a connector 634, thereby supplying vacuum to apparatus 600. Non-condensable gas stream 634 exits decanter 630 and is fed to vacuum pump 636. Aqueous 2-butanol stream 632 is then fed to a distillation apparatus (not shown) that is capable of further dehydrating the aqueous 2-butanol stream to produce a dry 2-butanol stream for use as the feed to a reaction vessel in which dry 2-butanol is catalytically converted to a reaction product comprising at least one dibutyl ether.

### General Methods and Materials

In the following examples, “C” is degrees Centigrade, “mg” is milligram; “ml” is milliliter; “temp” is temperature; “MPa” is mega Pascal; “GC/MS” is gas chromatography/mass spectrometry.

Amberlyst® (manufactured by Rohm and Haas, Philadelphia, Pa.), tungstic acid, 2-butanol, H₃PO₄ and H₂SO₄ were obtained from Alfa Aesar (Ward Hill, Mass.); CBV-3020E was obtained from PQ Corporation (Berwyn, Pa.); Sulfated Zirconia was obtained from Engelhard Corporation (Iselin, N.J.); 13% Nafion®/SiO₂ can be obtained from Engelhard; and H-Mordenite can be obtained from Zeolyst Intl. (Valley Forge, Pa.).

### General Procedure for the Conversion of 2-Butanol to Dibutyl Ethers

A mixture of 2-butanol and catalyst was contained in a 2 ml vial equipped with a magnetic stir bar. The vial was sealed with a serum cap perforated with a needle to facilitate gas exchange. The vial was placed in a block heater enclosed in a pressure vessel. The vessel was purged with nitrogen and the pressure was set at 6.9 MPa. The block was brought to the indicated temperature and controlled at that temperature for the time indicated. After cooling and venting, the contents of the vial were analyzed by GC/MS using a capillary column (either (a) CP-Wax 58 [Varian; Palo Alto, Calif.], 25 m×0.25 mm, 45 C/6 min, 10 C/min up to 200 C, 200 C/10 min, or (b) DB-1701 [J&W (available through Agilent; Palo Alto, Calif.)], 30 m×0.25 mm, 50 C/10 min, 10 C/min up to 250 C, 250 C/2 min).

The examples below were performed according to this procedure under the conditions indicated for each example.

### EXAMPLES 1-14

### Reaction of 2-Butanol (2-BuOH) with an Acid Catalyst to Produce Dibutyl Ethers

The reactions were carried out for 2 hours at 6.9 MPa of N₂.

| | | | | Dibutyl |
|---|---|---|---|---|
| Example | | Temp | 2-BuOH % | Ethers |
| Number | Catalyst (50 mg) | (C.) | Conversion | % Selectivity |
| 1 | H₂SO₄ | 120 | 67.5 | 8.8 |
| 2 | Amberlyst 15 | 120 | 31.2 | 3.7 |
| 3 | 13% Nafion/SiO₂ | 120 | 17.9 | 2.4 |
| 4 | CBV-3020E | 120 | 25.0 | 1.7 |
| 5 | H-Mordenite | 120 | 28.3 | 2.7 |
| 6 | Tungstic Acid | 120 | 28.0 | 2.1 |
| 7 | Sulfated Zirconia | 120 | 24.5 | 0.8 |
| 8 | H₂SO₄ | 200 | 75.6 | 0 |
| 9 | Amberlyst 15 | 200 | 26.8 | 0 |
| 10 | 13% Nafion/SiO₂ | 200 | 49.6 | 45.2 |
| 11 | CBV-3020E | 200 | 38.6 | 40.2 |
| 12 | H-Mordenite | 200 | 48.5 | 57.2 |
| 13 | Tungstic Acid | 200 | 24.4 | 29.0 |
| 14 | Sulfated Zirconia | 200 | 8.6 | 5.2 |

### EXAMPLES 15-19

### Reaction of 2-Butanol (2-BuOH) with an Acid Catalyst in the Presence of Trimethylpentane (TMP) to Produce Dibutyl Ethers

The reactions were carried out for 2 hours at 120 C and 6.9 MPa of N₂. The feedstock was 30% 2-butanol by weight (relative to the total weight of the 2-butanol plus TMP).

| Example | | 2-BuOH | Ethers |
|---|---|---|---|
| Number | Catalyst (50 mg) | % Conversion | % Selectivity |
| 15 | H₂SO₄ | 98.9 | 5.0 |
| 16 | H₃PO₄ | 50.5 | 2.3 |
| 17 | Amberlyst ® 15 | 76.9 | 2.6 |
| 18 | 13% NAFION ® | 16.9 | 12.6 |
| 19 | CBV-3020E | 19.6 | 8.8 |

1. A process for making at least one dibutyl ether comprising:
(a) obtaining a fermentation broth comprising 2-butanol;
(b) separating dry 2-butanol from said fermentation broth to form separated dry 2-butanol;
(c) contacting the separated dry 2-butanol of step (b), optionally in the presence of a solvent, with at least one acid catalyst at a temperature of about 50 degrees C. to about 450 degrees C. and a pressure from about 0.1 MPa to about 20.7 MPa to produce a reaction product comprising said at least one dibutyl ether; and
(d) recovering said at least one dibutyl ether from said reaction product to obtain at least one recovered dibutyl ether.

2. The process of claim 1, wherein said separating comprises the step of distillation.

3. The process of claim 2, wherein said separating further comprises at least one step selected from the group consisting of pervaporation, gas-stripping, adsorption, and liquid-liquid extraction.
